(19) United States
(12) Patent Application Publication
(10) Pub. No.: US 2011/0130371 A1
(43) Pub. Date: Jun. 2, 2011

(54) C-ring-substituted pregn-4-ene-21,17-carbolactones, and pharmaceutical preparations comprising the same

(75) Inventors: Sven Ring, Jena (DE); Rolf Bohlmann, Berlin (DE); Joachim Kuhnke, Potsdam (DE); Ludwig Zorn, Berlin (DE); Steffen Borden, Berlin (DE); Katja Prelle, Ratinjen (DE)

(73) Assignee: BAYER SCHERING PHARMA AKTIENGESSELSCHAFT, Berlin (DE)

(21) Appl. No.: 12/995,475

(22) PCT Filed: May 26, 2009
(86) PCT No.: PCT/EP2009/003716

§ 371 Date: Feb. 9, 2011

(30) Foreign Application Priority Data
Jun. 2, 2008 (DE) 10 2008 026 793.7

(51) Int. Cl. C07J 71/00; A61K 31/58; C12P 33/20

(52) U.S. Cl. 514/170; 540/23; 514/173; 435/53

(57) ABSTRACT

The present invention relates to C-ring-substituted pregn-4-ene-21,17-carbolactones of the general formula I

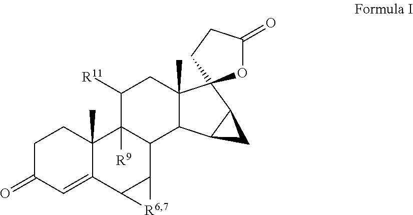

in which
R^{6,7} is an α- or β-methylene and
R⁹ is a hydrogen atom and R¹¹ is a bromine, chlorine or fluorine atom or
R⁹ and R¹¹ together are a bond.

The novel compounds are progestational antimineralocorticoids.

The present invention relates to C-ring-substituted pregn-4-ene-21,17-carbolactones of the general formula I

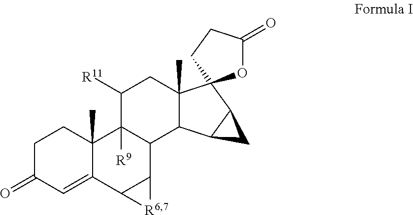

in which
R^{6,7} is an α- or β-methylene and
R⁹ is a hydrogen atom and R¹¹ is a bromine, chlorine or fluorine atom or
R⁹ and R¹¹ together are a bond.

The hydrogen atom R⁹ is preferably located in the α position.

The halogen atom R¹¹ is preferably located in the β position.

A fluorine or chlorine atom are preferred as halogen atom R¹¹; a fluorine atom is particularly preferred.

The compounds mentioned below are particularly preferred according to the invention: 11β-chloro-6β,7β;15β,16β-dimethylene-3-oxo-17-pregn-4-ene-21,17β-carbolactone 6β,7β;15β,16β-dimethylene-3-oxo-17-pregna-4,9(11)-diene-21,17β-carbolactone 6β,7β;15β,16β-dimethylene-11β-fluoro-3-oxo-17-pregn-4-ene-21,17β-carbolactone 6α,7α;15β,16β-dimethylene-11β-fluoro-3-oxo-17-pregn-4-ene-21,17β-carbolactone drospirenone(6β,7β-15β,16β-dimethylene-3-oxo-17-pregn-4-ene-21,17β-carbolactone) is a new progestogen which is present for example in the oral contraceptive YASMIN® and the product ANGELIQ® for the treatment of postmenopausal symptoms. Owing to its comparatively low affinity for the progestogen receptor and its comparatively high ovulation-inhibitory dose, drospirenone is present in YASMIN® in the relatively high daily dose of 3 mg.

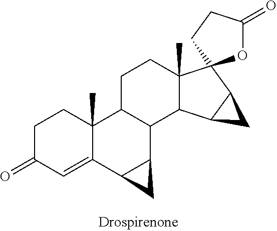

Drospirenone is notable for having, in addition to the progestational effect, an aldosterone-antagonistic (antimineralocorticoid) and antiandrogenic effect. These two properties make drospirenone very similar to the natural progestogen progesterone in its pharmacological profile but, unlike drospirenone, the latter has insufficient oral bioavailability.

It is therefore an object of the present invention to provide compounds which are intended to have a higher progestational potency than drospirenone in vivo. This is ultimately intended to be manifested by a lower daily dosage and to lead to a lower active compound substance requirement.

The compounds to be provided by the present invention are intended additionally to have an antimineralocorticoid effect in vivo which at most is as high as that of drospirenone but preferably is less than the latter.

It is further intended that the compounds of the invention have a weaker antiandrogenic activity than drospirenone.

Finally, the compounds of the invention are intended to have high metabolic stability.

WO 2006072467 discloses compounds which show an activity in the pregnancy maintenance test on rats which is much higher than that of drospirenone and show an activity on the mineralocorticoid receptor from rat kidney homogenate which is comparable to that of drospirenone. These compounds are 18-methyl-19-nor-17-pregn-4-ene-21,17-carbolactones.

Compounds having in vitro a less dissociated profile than drospirenone in relation to their binding to the progesterone and mineralocorticoid receptors are described in WO 2008000521, These compounds are 18-methyl-19-nor-androst-4-ene-17,17-spiro ethers.

A process for preparing 3-oxopregn-4-ene-21,17-carbolactones by metal-free oxidation of 17-(3-hydroxypropyl)-3,17-dihydroxyandrostanes is described in EP 1 746 101 A1. A pharmacological activity is not generally evident from EP 1 746 101 A1 for these carbolactones. The only specific compound mentioned is 6β,7β-15β,16β-dimethylene-3-oxo-17-pregn-4-ene-21,17β-carbolactone(drospirenone). 11-Halo and 9,11-dehydro compounds are specifically not shown.

The object of the present invention is achieved through the provision of the C-ring-substituted pregn-4-ene-21,17-carbolactones of the general formula 1 described herein. The compounds of the general formula I (and especially those of Example 1 and 2) are distinguished by an improved profile of effects.

The compounds of the invention are notable for a surprisingly strong Progestational activity and have high activity in the pregnancy maintenance test on rats after subcutaneous administration.

The compounds of the invention of the general formula 1 have a greater progestational activity with, at the same time, weaker binding to the androgen receptor than drospirenone.

It has additionally been found that the compounds of the invention show a potassium-retaining natriuretic (antimineralocorticoid) effect in adrenalectomized rats.

Owing to their progestational activity, the novel compounds of the general formula I can be used alone or in combination with oestrogen in pharmaceutical products for contraception.

Thus, the compounds of the invention of the general formula can be used alone, i.e. without oestrogen, for producing so-called POPS (progesterone-only pill). Such POPs based on other compounds with progestational activity have been disclosed, for example based on the progestogen levonorgestrel in the form of the product Microlut® (28 daily dose units each comprising 30 μg of levonorgestrel).

Because of their favourable profile of effects, the compounds of the invention are particularly suitable for the treatment of premenstrual symptoms such as headaches, depressive moods, water retention and mastodynia.

The compounds of the invention are, owing to their progestational activity, suitable for further possible uses as are generally known for progestogens, for example the treatment of severe bleeding disorders, for example of menorrhagias and metrorrhagias, treatment of corpus luteum insufficiency, i.e. treatment of threatened abortion, treatment of delayed puberty and treatment of conditions which make progestogen replacement appear indicated.

The present invention therefore also relates to pharmaceutical products which comprise at least one compound of the general formula 1 together with a pharmaceutically acceptable carrier.

Pharmaceutical products preferred according to the invention are those comprising 6β,7β;15β,16β-dimethylene-3-oxo-17-pregna-4,9(11)-diene-21,17β-carbolactone or 6β,7β;15β,16β-dimethylene-11β-fluoro-3-oxo-17-pregn-4-ene-21,17β-carbolactone as active ingredient.

The present invention also relates to pharmaceutical combination products which, besides a compound of the general formula I and the pharmaceutically acceptable carrier, comprise an oestrogen.

The dosage of the compounds of the invention in contraceptive products is intended to be from 0.01 to 5 mg, preferably 0.01 to 2 mg, per day.

The daily dose for the treatment of premenstrual symptoms is about 0.1 to 20 mg.

The progestational and oestrogenic active ingredient components are preferably administered orally together in contraceptive products. The daily dose is preferably administered all at once.

Suitable oestrogens in the combination products of the invention for contraception are oestradiol and synthetic oestrogens, preferably ethinylestradiol, but also mestranol. It is additionally possible to use esters of oestradiol, and of these in particular oestradiol valerate or else oestradiol benzoate.

The oestrogen is administered in a daily amount corresponding in its oestrogenic effect to that of from 0.01 to 0.04 mg of ethinylestradiol. Ethinylestradiol itself is used in a daily amount of from 0.01 to 0.04 mg in such contraceptive products.

The novel compounds of the general formula I can also be employed in pharmaceutical products for treating pre-, pen- and post-menopausal symptoms and in products for hormone replacement therapy (HRT).

Oestrogens used in products of this type for hormone replacement therapy are primarily natural oestrogens, especially oestradiol or its esters, for example oestradiol valerate or else conjugated oestrogens (CEEs=conjugated equine estrogens) as are present for example in the product PREMARIN®.

There have also been descriptions recently of incorporating folic acid (WO 99/53910) or 5-methyl-6-(S)-tetrahydrofolate, and of these in particular the calcium salt of 5-methyl-6-(S)-tetrahydrofolic acid (Metafolin®; WO 2006/120035), into products for contraception or hormone replacement therapy:

Corresponding stable formulations of tetrahydrofolates with a progestogen alone and in particular with a progestogen and with an oestrogen are described in WO 2008/003432. In the case of products for contraception, folic acid or the tetrahydrofolate component serves to prevent malformation of the maturing foetus. The priority in this connection is to prevent NTDs (Neural Tube Defects) in the neonate, a serious physical malformation.

It is within the scope of the present invention to employ the novel progestogens of the invention in analogy to the description in the above publications for previously known progestogens.

The pharmaceutical products based on the novel compounds are formulated in a manner known per se by processing the active ingredient, where appropriate in combination with an oestrogen, with the carrier substances, diluents, where appropriate masking flavours etc., common in pharmaceutical technology, and converted into the desired administration form.

If the novel compounds are to be used, alone or together with an oestrogen, jointly with folic acid or with a 5-methyl-6-(S)-tetrahydrofolate, corresponding formulations can be produced as described for previously known progestogens in the above publications.

Suitable for the preferred oral administration are in particular tablets, coated tablets, capsules, pills, suspensions or solutions.

Suitable for parenteral administration are in particular oily solutions such as, for example, solutions in sesame oil, castor oil and cottonseed oil. It is possible to add solubilizers such as, for example, benzyl benzoate or benzyl alcohol to increase the solubility.

It is also possible to incorporate the substances of the invention in a transdermal system and administer them transdermally therewith.

It is likewise possible for the novel compounds to be incorporated, alone or jointly with an oestrogen, into an administration system which releases the active ingredient or active ingredients over a prolonged period, for example an intrauterine system (IUS), an intravaginal ring (IVR) or into a system which is implanted under the skin, from which they are gradually released after insertion thereof into the uterus or vagina or the implantation underneath the skin.

Pharmacology

Progestational Effect in Ovariectomized Rats:

The progestational effect was determined as described by Muhn et al. (Muhn, P., Krattenmacher, R., Beier, S., Eiger, W., and Schillinger, E. (1995). Drospirenone: a novel progestogen with antimineralocorticoid and antiandrogenic activity. Pharmacological characterization in animal models. Contraception 51, 99-110).

This entails investigating the ability of the compounds to compensate for the lack of progesterone in ovariectomized animals which no longer have their own progesterone synthesis and to maintain pregnancy (“pregnancy maintenance”).

Female animals weighing 200-230 g were mated. The animals were ovariectomized on day 8 post coitum (p.c.) and treated with 5 μg/kg/d oestrone. The compounds to be tested were given in various concentrations (3, 10, 30 mg/kg/d). Treatment was started on day 8 p.c. and continued for 6 days.

Evaluation:

One day after the last treatment, the animals were autopsied. The degree of pregnancy maintenance was calculated by dividing the number of live fetuses by the number of detectable implantation sites. The presence of a beating heart was decisive for assessing a fetus as alive. No identifiable implantation sites (ovariectomized controls) was defined as 0% pregnancy maintenance.

The ED50 (concentration at which the half-maximum effect occurs) was determined as a measure of the progestational potency.

It was found that the compounds of the invention have a progestational potency which is up to four times higher than the progestational effect of drospirenone.

TABLE 1
Progestational effect of the compounds
in the pregnancy maintenance test.

| | ED₅₀ |
|---|---|
| Drospirenone | 10 mg/kg/d |
| Compound of Ex. 1 | 2.9 mg/kg/d |
| Compound of Ex. 2 | 3.4 mg/kg/d |

Progestational Effect in Ovariectomized Rats, Correlation of Pharmacokinetic Values and Pharmacodynamic Effects

In order to describe the difference in their progestational potency more precisely, in a modified pregnancy maintenance experiment blood samples were additionally taken at various times to determine pharmacokinetic parameters. The rats were pretreated in this case as described in the preceding experiment. A physiologically based pharmacokinetic model was developed using the programmes PK-SIM, version 4.0.1 (Bayer Technology Services, Leverkusen, Germany), GastroPlus version 5.2 (Simulations Plus, Inc., Lancaster, Calif., USA) and WinNonlin® Professional (version 5.2, Pharsight Corp., Mountain View, Calif., USA).

The relation was found between the AUC (area under the curve of the time-concentration profile) reached during the experiment and the measured pharmacological effect, i.e. the pregnancy maintenance achieved. The model thus permitted estimation of AUC₅₀ values.

A statistically significant difference in the progestational in vivo potencies between drospirenone and the compounds of the invention was found with AUC₅₀ values more than 6 times lower for the compounds of the invention. Based on the relation between systemic exposure and effect, the AUC in blood necessary to achieve 80% pregnancy maintenance in the rat model was calculated (Table 2).

TABLE 2
Exposure necessary to achieve pregnancy maintenance (calculated)

| | AUC (blood, calculated) in rats necessary |
|---|---|
| | for 80% pregnancy maintenance |
| Drospirenone | 1346 (μg*h/L) |
| Compound of Ex. 1 | 210 (μg*h/L) |

Antimineralocorticoid Effect of the Compounds in Adrenalectomized Rats (Diuresis Experiments):

The antimineralocorticoid effect of the compounds was determined as described by Losert at al. (Losert, W., Casals-Stenzel, J., and Buse, M. (1985). Progestogens with antimineralocorticoid activity. Arzneimittelforschung 35, 459-471).

Male animals weighing 180-200 g were adrenalectomized 5 days before the experiment and received replacement with glucocorticoids. A diuresis experiment was carried out on day 5 after the adrenalectomy. A continuous infusion of isotonic NaCl solution plus 5% glucose was administered i.v. to the animals. Simultaneous administration of 1 μg/kg/h d-aldosterone achieved a constant mineralocorticoid effect, identifiable from sodium retention and kaliuresis. The test compounds were administered s.c. in various dosages (3, 10 and 30 mg/kg), and the abolition of the aldosterone-induced sodium retention indicates an antimineralocorticoid effect.

Evaluation:

The animals were kept in metabolism cages and urine fractions were collected each hour. The sodium and potassium ion concentration in the urine was determined by a flame-photometry method, and the Na/K ratio was calculated therefrom. The Na/K ratios were plotted against time and the area under the curve [AUC] was determined. The ED₅₀ (concentration at which the half-maximum effect occurs) was determined as a measure of the antimineralocorticoid potency.

It was found that the compounds of the invention have antimineralocorticoid activity which is about half as strong to about as strong as that of drospirenone.

TABLE 3
Antimineralocorticoid effect of the
compounds in the diuresis experiment.

| | ED₅₀ | |
|---|---|---|
| Drospirenone | 6.7 | mg/kg |
| Compound of Ex. 1 | 5.9 | mg/kg/d |
| Compound of Ex. 2 | 12.36 | mg/kg/d |

Antiandrogenic Effect of the Compounds In Vitro in the Antiandrogen Transactivation Assay:

The antiandrogenic effect was carried out as described in Schneider et al. (Schneider K., Graf E., Irran E., Nicholson G., Stainsby F. M., Goodfellow M., Borden S. A., Keller S., Süssmuth R. D. and Fiedler H. P. (2008) Bendigoles A˜C, New Steroids from Gordonia australis Acta 2299, J. Antibiotics (Tokyo) 61 (6), 356-364).

The culture medium used for culturing cells used for the assay was RPMI (PAA, #E15-49) with 10% FCS, 200 mM L-glutamine, 100 U/100 ug/ml penicillin/streptomycin. Reporter cell lines (PC3 cells stably transfected with human androgen receptor (hAR) and a reporter construct which comprises luciferase under the control of an androgen-responsive promoter (MMTV)) were grown at a density of 4×10⁴ cells per well in white, opaque tissue culture plates with 96 wells in each (Perkin Elmer, #P12-106-017) and maintained in culture medium with 3% DCC-FCS (serum treated with activated carbon to remove interfering components present in the serum). The compounds to be investigated were added eight hours later, and the cells were incubated with the compounds for 16 hours. The experiments were carried out in triplicate. At the end of the incubation, the effector-containing medium was removed and replaced by lysis buffer.

After luciferase assay substrate (Promega, #E1501) had been added, the plates with the 96 wells were then inserted into a microplate luminometer (Pherastar, BMG labtech), and the luminescence was measured. The IC₅₀ values were evaluated using software for calculating dose-activity relationships. The efficacy indicates the per cent of the maximum effect by comparison with the maximum effect of a reference antiandrogen (hydroxyflutamide).

It was found that the compounds of the invention have weaker antiandrogenic activity than drospirenone in vitro.

TABLE 4
Antiandrogenic effect of the compounds
in the transactivation assay.

| | IC₅₀ [μM] | Efficacy |
|---|---|---|
| Drospirenone | 0.12 | 40.3 |
| Compound of Ex. 1 | 0.24 | 19.3 |
| Compound of Ex. 2 | 0.16 | 31.9 |

Antiandrogenic Effect of the Compounds in Orchidectomized Rats (Hershberder):

The antiandrogenic effect of the compounds was determined as described in Muhn et al. (Muhn, P., Krattenmacher, R., Beier, S., Eiger, W., and Schillinger, E. (1995). Drospirenone: a novel progestogen with antimineralocorticoid and antiandrogenic activity. Pharmacological characterization in animal models. Contraception 51, 99-110). This entails testing the suitability of the compounds for inhibiting the androgen-dependent growth of prostate, seminal vesicle and levator ani muscle in young, male, castrated rats receiving androgen replacement.

For this purpose, young rats are initially castrated. Eight days after the orchidectomy, the animals receive 1 mg/kg/day testosterone propionate (TP) s.c. alone or in combination with the test substances (10 mg/kg/day) for seven days.

On day 15 after the orchidectomy, the animals are sacrificed, and prostate, seminal vesicle and levator ani muscle are dissected out, and the relative wet weight is determined. The inhibited androgen-induced growth serves as a measure of the antiandrogenic effect of the test substance. The antiandrogenic effect was converted into per cent inhibition, with full effect (100% inhibition) when the prostate weight corresponded to the vehicle control, and 0% inhibition when the prostate weight corresponded to the TP treatment.

TABLE 5
Antiandrogenic effect of the compounds in orchidectomized rats

| | Antiandrogenic effect (% inhibition) |
|---|---|
| Drospirenone | 22.5% |
| Compound of Ex. 1 | <0% |
| Compound of Ex. 2 | 3.8% |
| Cyproterone acetate | 73.1% |

In agreement with their clinical profile it was found that cyproterone acetate shows a strong antiandrogenic effect and drospirenone a smaller but distinct antiandrogenic effect. It was found in contrast thereto that the compounds of the invention show no antiandrogenic effect.

Antiandrogenic Effect of the Compounds in Orchidectomized Rats, Investigation of Gene Expression

As a modification of the Hershberger assay described above, in addition animals were sacrificed 24 hours after the first treatment (1 mg/kg TP, 10 mg/kg test substance), and prostate tissue was shock-frozen immediately after the autopsy and then employed for mRNA isolation. A quantitative PCR method (TagMan) was used to investigate the induction (x-fold induction factor) of androgen-stimulated genes, inter alis of steroid biosynthesis (e.g. IDI1, NM 004508.2) as a measure of the androgenic effect of TP and the inhibition thereof by the compounds of the invention. The inhibition was converted into % inhibition, with complete inhibition (100%) when the induction factor is 1, and 0% inhibition when the induction factor is that of TP.

TABLE 6
Antiandrogenic effect of the compounds:
alteration in gene expression

| | x-fold induction factor of | Antiandrogenic |
|---|---|---|
| | IDI1 mRNA after TP | effect |
| | administration | (% inhibition) |
| Control (TP) | 7.07 | 0% |
| TP + DRSP | 5.39 | 27% |
| TP + Ex. 1 | 7.89 | <0% |
| TP + CPA | 1.53 | 91% |

TP = testosterone propionate; DRSP = drospirenone; CPA = cyproterone acetate

In agreement with their clinical profile it was found that cyproterone acetate shows a strong antiandrogenic effect and drospirenone a smaller but distinct antiandrogenic effect. It was found in contrast thereto that the compound of Ex. 1 of the invention shows no antiandrogenic effect.

The novel compounds of the general formula I are prepared according to the invention as described below. The synthesis route for the novel C-ring-substituted pregn-4-ene-21,17-carbolactones shown in Scheme 1 starts for example from the known compound 1 [CAS: 95218-07-s, Nickisch et al. J. Med. Chem. 1985, 546-550].

Introduction of a Δ^{9,11} double bond for example by mesylation and elimination [Chamberlin et al. J. Org. Chem. 1960, 295] affords compound 2 (Example 1).

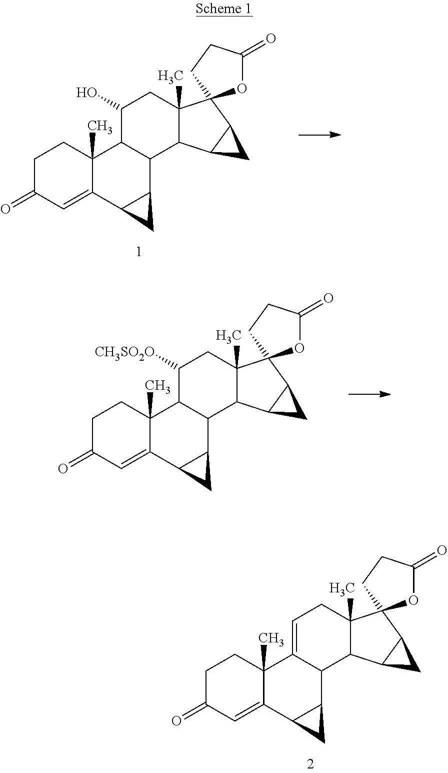

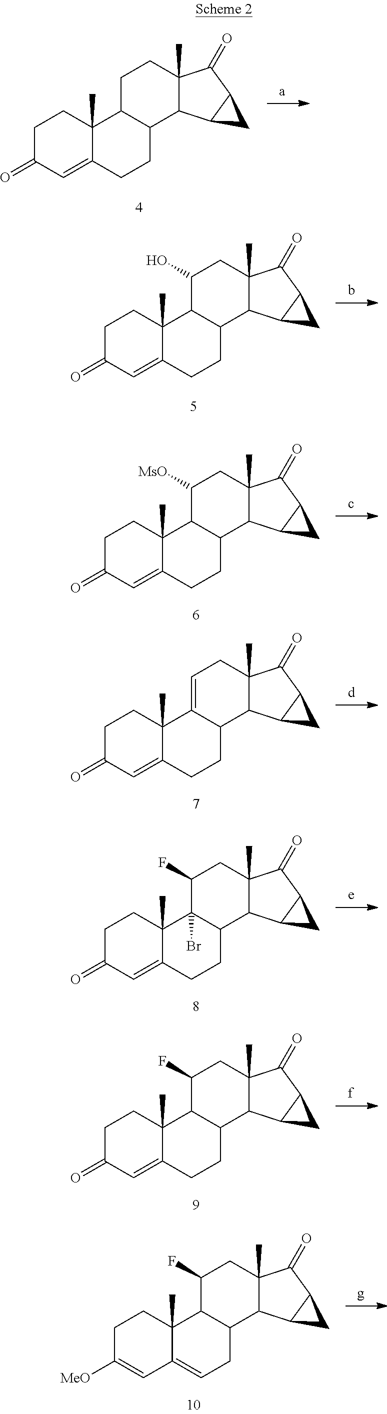

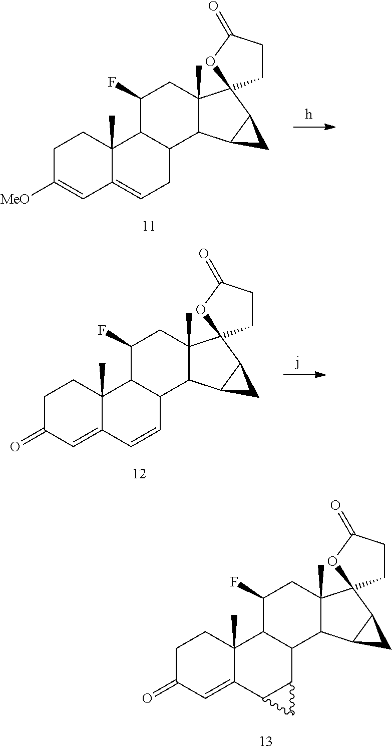

Conditions: a) Aspergillus ochraceus; b) CH₃SO₂Cl, pyridine, DMAP; c) NaOAc, AcOH, Ac₂O; d) dibromodimethyihydantoin, HF-pyridine (70%), dichloromethane; e) Bu₃SnH/AlBN/benzene; f) 2,2-dimethoxypropane, pyridinium-pTsOH; g) CH₂═CHCH₂OPO(NMe₂)₂, n-BuLi, THF; h) 1. N-methylpyrrolidone, NaOAc/H₂O, dibromodimethylhydantoin, 2. LiBr/Li₂CO₃; i) (CH₃)₃SO—I, DMSO, NaH.

Another process for preparing the compound of the invention is shown in Scheme 2.

(5) is obtained from the known 15β,16β-methyleneandrost-4-ene-3,17-dione [Wiechert et at Chem. Ber. 106, 1973, 888] (4) by microbiological hydroxylation in a fermentor with microorganisms which bring about hydroxylation of the steroid in the 11 position, in particular in the 11α position, e.g. of the species Absidia sp., Acremonium sp., Ascochyta sp., Aspergillus sp., Bacillus sp., Beauveria sp., Botryodipoldia sp., Caldariomyces sp., Calonectria sp., Colletotrichum sp., Curvularia sp., Fusarium sp., Gibberella sp., Gloeosporium sp., Glomerella sp., Gnomonia sp., Haplosporella sp., Helicostylum sp., Helminthosporium sp., Metarhizium sp., Mucor sp., Nigrospora sp., Rhizopus sp., Sporotrichum sp., Syncephalastrum sp., and Wojnowicia sp.

Use is made in particular of Absidia orchidis, Absidia coerulea, Acremonium strictum, Ascochyta clematidina, Aspergillus alliaceus, Aspergillus awamori, Aspergillus fischeri, Aspergillus flavus, Aspergillus malignus, Aspergillus melleus, Aspergillus nidulans, Aspergillus niger, Aspergillus ochraceus, Aspergillus variecolor, Bacillus megaterium, Beauveria bassiana, Beauveria tenella, Botryodiplodia malorum, Caldariomyces fumago, Calonectria decora, Colletotrichum phomoides, Curvularia lunata, Fusarium oxysporium, Fusarium solani, Gibberella zeae, Glomerella cingulata, Gloeosporium fructigenum, Gloeosporium higgensianum, Gloeosporium kaki, Gloeosporium lacticolor, Gloeosporium olivarum, Glomerella fusaroides, Gnomonia cingulata, Haplosporella hesperedica, Helminthosporium sp., Helicostylum piriforme, Metarhizium anisopliae, Mucor plumbeus, Mucor spinosus, Nigrospora sphaerica, Rhizopus arrhizus, Rhizopus cohnii, Rhizopus delemar, Rhizopus japonicus, Rhizopus kazaensis, Rhizopus microsporus, Rhizopus oryzae, Rhizopus shanghaiensis, Rhizopus stolonifer, Rhizopus tritici, Sporotrichum sulfurescens, Syncephalastrum racemosum, Wojnowicia graminis and Wojnowicia hirta.

Those particularly employed are Absidia orchidis (ATCC 6647), Acremonium strictum (NRRL 5759), Ascochyta clematidina (CBS), Aspergillus alliaceus (ATCC 10060), Aspergillus awamori (CBS), Aspergillus fischeri (ATCC 1020), Aspergillus malignus (IMI 16061), Aspergillus melleus (CBS), Aspergillus nidulans (ATCC 11267), Aspergillus niger (ATCC 9142, ATCC 11394), Aspergillus ochraceus (NRRL 405, NRRL 410, CBS 13252, ATCC 46504), Aspergillus variecolor (ATCC 10067), Bacillus megaterium (ATCC 13368), Beauveria bassiana (IFO 5838, ATCC 13144, IFO 4848, CBS 11025, CBS 12736, ATCC 7159), Botryodiplodia maiorum (CBS 13450), Caldariomyces fumago (ATCC 16373), Calonectria decora (ATCC 14767), Curvularia lunata (IX 3, NRRL 2380), Fusarium solani (ATCC 12823), Fusarium oxysporum (ATCC 7808), Gibberelia zeae (CBS 4474), Glomerella cingulata (ATCC 12097, ATCC 10534, CBS 23849, CBS 23749, ATCC 16646, IFO 6459, IFO 6425, IFO 6470, ATCC 15093, ATCC 10529, IFO 5257, ATCC 56596, ATCC 64682), Glomerella fusaroides (ATCC 9552), Gnomonia cingulata (CBS 15226), Haplosporella hesperedica (CBS 20837), Helicostylum piriforme (ATCC 8992), Helminthosporium sp. (NRRL 4671), Metarhizium anisopliae (IFO 5940), Mucor plumbeus (CBS 29563), Nigrospora sphaerica (ATCC 12772), Rhizopus arrhizus (ATCC 11145), Rhizopus oryzae (ATCC 4858, ATCC 34102, CBS 32947), Rhizopus stolonifer (ATCC 15441), Syncephalastrum racemosum (IFO 4827) and Wojnowicia graminis (CBS 89168).

The 11-hydroxysteroid 5 is then converted for example by mesylation and basic elimination of the methanesulphonic acid into the Δ⁹⁽¹¹⁾ derivative 7. The latter can be converted for example by a bromofluorination of the Δ⁹⁽¹¹⁾ double bond by known processes, e.g. with Olah's reagent/N-bromosuccinitnide [Olah et al. Synthesis 1973, 780] into the dione 8, and converted by reduction debromination, e.g. with tributyltin hydride, into the fluoro dione 9. After protection of the 4-en-3-one system (9) as dienol ether 10, the spirolactone is established for example by the method of Sturtz [Synthesis 1980, 289] or alternatively by known processes [Bittler Angew. i.e. 21 1982, 696; Laurent J. Steroid Biochem. 19 1983, 771]. Compound 11 can be converted for example by dienol ether bromination in analogy to the method of [J. A. Zderic, Humberto Carpio, A. Bowers and Carl Djerassi Steroids 1 1963, 233] and hydrogen bromide elimination by heating the 6-bromo compound with basic reagents such as, for example, LiBr or Li₂CO₃ in aprotic solvents such as, for example, dimethylformamide or 1-methyl-2-pyrrolidone at temperatures of 50-120° C. or else by heating the 6-bromo compounds in a solvent such as collidine or lutidine, into the 4,6-dien-3-one 12. Compound 12 is then converted by methenylation of the Δ⁶ double bond by known processes, e.g. with dimethylsulphoxonium methylide [see, for example, DE-A 11 83 500, DE-A 29 22 500, EP-A 0 019 690, U.S. Pat. No. 4,291,029; E. J. Corey and M. Chaykovsky, J. Am. Chem. Soc. 84 1962, 867] into a compound 13, resulting in a mixture of the α and β isomers (the ratio depends on the substrates used, with the β isomer usually distinctly predominating), which can be separated into the individual isomers for example by chromatography. Introduction of an 11-fluoro group can also take place as shown in Scheme 3 for example starting from an 11-hydroxy 4-enedione 5 by reaction with nonaflyl fluoride and DBU in an organic solvent, e.g. tetrahydrofuran [see, for example, Bennua-Skalmowski, Tet. Lett. 1995, 2611] to form a mixture of the abovementioned 11-fluorosteroide 9 and of the likewise abovementioned Δ⁹⁽¹¹⁾ derivative 7, which can be separated into the individual compounds by chromatography, and subsequently be reacted further as described above.

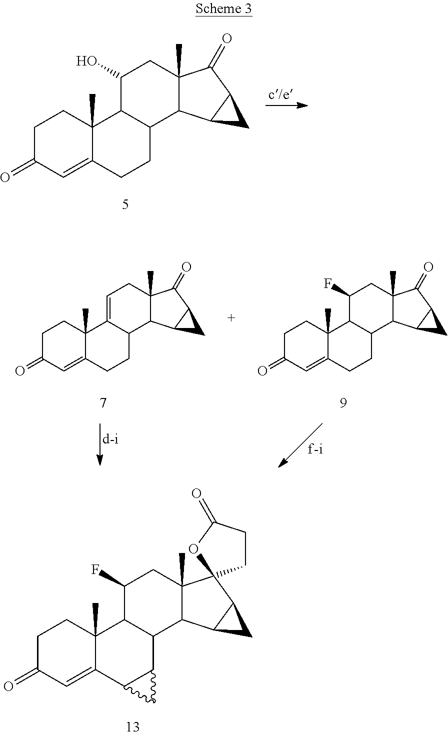

Conditions: c′/e′) NfF, DBU, THF; d-i) see Scheme 2.

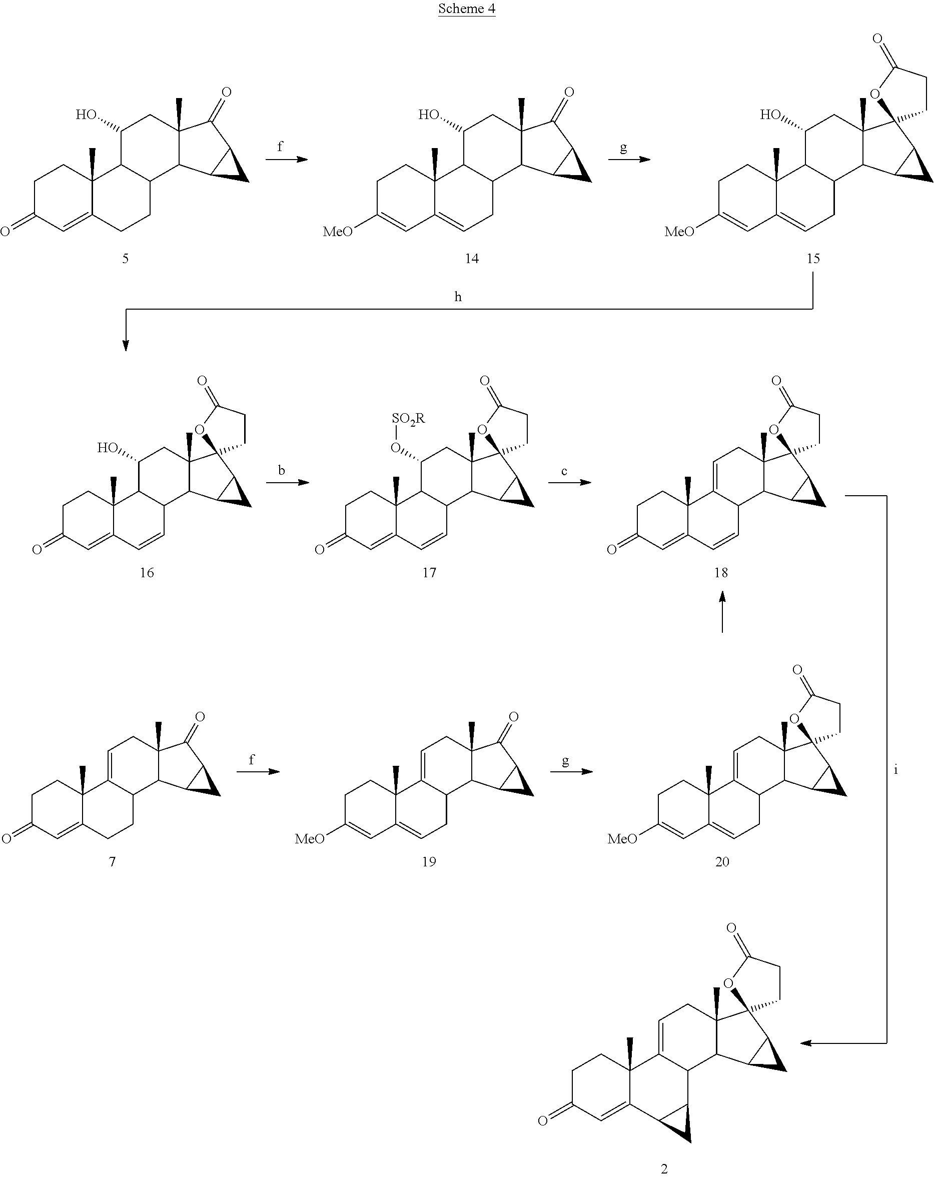

See Scheme 2 (R=methyl, tolyl) for all reaction conditions

Starting from 11-hydroxysteroid 5 it is possible in 5 stages to prepare the 4,6-dien-3-one 18 by methods described above; from which the compound 2 of the invention is subsequently obtained by methenylation of the 6,7 double bond (see above). The 4,6-dien-3-one 18 can also be obtained starting from the 4-en-3-one 7 in three stages by methods described above.

The intermediate compounds of the formulae 5, 6, 7, 8, 9, 10, 11, 12, 14, 15, 16, 17, 18, 19 and 20 are all new compounds. The present invention therefore relates to all of them. The present invention additionally relates to the use thereof as starting compounds and intermediates for the preparation of the compounds of the invention of the general formula I.

The following examples serve to explain the invention in more detail:

### EXAMPLE 1

### 6β,7β;15β,16β-Dimethylene-3-oxo-17-pregna-4,9(11)-diene-21,17β-carbolactone

### a) 6β,7β;15β,16β-Dimethylene-11α-mesyloxy-3-oxo-17-pregn-4-ene-21,17β-carbolactone

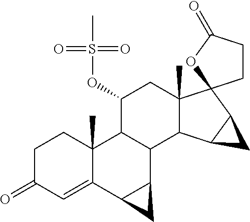

21.7 ml of mesyl chloride were added dropwise to a solution of 25 g of 6β,7β;15β,16β-dimethylene-11α-hydroxy-3-oxo-17-pregn-4-ene-21,17β-carbolactone [CAS: 95218-07-8, Nickisch et al. J. Med. Chem. 1985, 546-550] in 250 ml of pyridine at 0° C., and the mixture was stirred at 25° C. for 2 hours. It was then diluted with ethyl acetate, washed with sodium bicarbonate solution, water and brine until neutral, dried over sodium sulphate, and concentrated in vacuo at 40° C. 30 g of pure 6β,7β;15β,16β-dimethylene-11α-mesyloxy-3-oxo-17-pregn-4-ene-21,17β-carbolactone were obtained as a solid.

¹H-NMR (600 MHz, CDCl3): δ=6.01 (s, 1H), 5.46 (d(br), 1H), 2.69-2.61 (m, 2H), 2.56 -2.46 (m, 3H), 2.39 (m, 1H), 2.27 (d(br), 1H), 2.19-2.13 (m, 2H), 1.89 (m, 1H), 1.81 (dd(br), 1H), 1.72 (m, 1H), 1.67 (m, 1H), 1.52-1.45 (m, 2H), 1.37 (m, 1H), 1.31 (m, 1H), 1.28 (s, 3H), 1.06 (m, 1H), 0.94 (s, 3H), 0.58 (m, 1H).

### b) 6β,7β;15β,16β-Dimethylene-3-oxe-17-pregna-4,9(11)-diene-21,17β-carbolactone

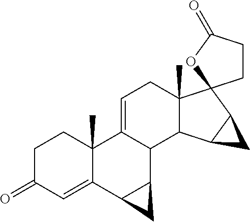

0.5 ml of acetic anhydride was added to a solution of 18.5 g of 6β,7β;15β,16β-dimethylene-11α-mesyloxy-3-oxo-17-pregn-4-ene-21,17β-carbolactone in 50 ml of acetic acid at 25° C., and the mixture was stirred at a bath temperature of 100° C. for 8 hours. It was then added to water, extracted three times with ethyl acetate, washed with water and brine until neutral, dried over sodium sulphate, and concentrated in vacuo at 40° C. 15.2 g of crude 6β,7β;15β,16β-dimethylene-3-oxo-17-pregna-4,9(11)-diene-21,17β-carbolactone were obtained. Chromatrography on silica gel with hexane/ethyl acetate resulted in 7.5 g of pure product as solid.

MS (EI): m/z=364 (M⁺),

¹H-NMR (400 MHz, CDCl3): δ=6.07 (s, 1H), 4.96 (m, 1H), 2.97 (s, 3H), 2.75-2.51 (m, 3H), 2.47-2.31 (m, 3H), 2.22-1.84 (m, 5H), 1.77-1.41 (m, 8H), 1.32-1.23 (m, 2H), 1.03 (s, 3H), 0.84 (m, 1H).

### EXAMPLE 2

### 6β,7β;15β,16β-Dimethylene-11β-fluoro-3-oxo-17-pregn-4-ene-21,17β-carbolactone

### a) 11α-Hydoxy-15β,16β-methyleneandrost-4-ene-3,17-dione

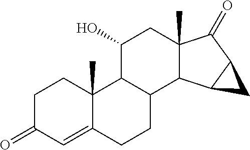

A 2 I Erlenmeyer flask containing 1 I of a nutrient solution, which had been sterilized in an autoclave at 121° C. for 30 minutes, of 3% glucose monohydrate, 1% corn steep liquor, 0.2% sodium nitrate, 0.1% potassium dihydrogen phosphate, 0.2% dipotassium hydrogen phosphate, 0.05% potassium chloride, 0.05% magnesium sulphate heptahydrate and 0.002% iron(II) sulphate heptahydrate (adjusted to pH 6.0) was inoculated with a 2 ml DMSO-ice culture of the strain Aspergillus ochraceus (NRRL 405) and shaken at 27° C. on a rotary shaker at 165 revolutions per minute for 71.5 hours. This preculture was used to inoculate a 20 I fermenter which was charged with 19 I of sterile medium of the same final composition as described for the preculture. In addition, before the sterilization, 1.0 ml silicone oil and 1.0 ml of Synperonic for foam control were also added. This fermenter was incubated under a superatmospheric pressure of 0.7 bar, at a temperature of 28° C., with aeration at 8 I per minute and with a stirring speed of 350 revolutions per minute for 47.5 hours.

2.5 I of preculture were removed from this 20 I fermenter in order to inoculate a 50 I fermenter which was charged with 47.5 I of sterile medium of the same final composition as described for the preculture. Before the sterilization, 2.5 ml of silicone oil and 2.5 ml of Synperonic were added. After an initial growth phase of 10 hours under a superatmospheric pressure of 0.7 bar, at a temperature of 28° C., with aeration at 10 I per minute and with a stirring speed of 350 revolutions per minute, a solution of 10.0 g of 15β,16β-methyleneandrost-4-ene-3,17-dione in 200 ml of DMF was added. Stirring was continued with aeration. The culture broth was harvested after 26 hours.

5.0 I of preculture were removed from the 20 I fermenter in order to inoculate a 100 I fermenter which was charged with 95.0 I of sterile medium of the same final composition as described for the preculture. Before the sterilization, 5.0 ml of silicone oil and 5.0 ml of Synperonic were added. After an initial growth phase of 10 hours under a superatmospheric pressure of 0.7 bar, at a temperature of 28° C., with aeration at 20 I per minute and with a stirring speed of 350 revolutions per minute, a solution of 20.0 g of 15β,16β-methyleneandrost-4-ene-3,17-dione in 400 ml of DMF was added. Stirring was continued with aeration. The culture broth was harvested after 26.25 hours.

The two culture broths were combined and extracted with 60 I of methyl isobutyl ketone for 19.75 hours. The combined organic phases were concentrated to dryness. The residue was washed with hexane in order to remove the silicone oil. The product was then crystallized from acetone, and 19.2 g (61% of theory) of 11α-hydroxy-15β,16β-methyleneandrost-4-ene-3,17-dione were isolated.

100 mg were purified by preparative HPLC (250×40 mm, Luna C18, 10 μ, 100 A, water-acetonitrile 70:30, 100 ml/min).

M.o.: 225/247-249° C.

[α]_{D}=+48.6° (CHCl₃, c=1.0700)

¹H-NMR (400 MHz, CDCl3): δ=1.04 (s, 3H), 1.13-1.37 (m, 8H), 1.62 (dt, 1H), 1.77-1.91 (m, 2H), 1.99 (m, 1H), 2.03-2.21 (m, 4H), 2.31-2.57 (m, 5H), 4.05 (m, 1H), 5.78 (s, 1H).

### b) 11α-Mesyloxy-15β,16β-methyleneandrost-4-ene-3,17-dione

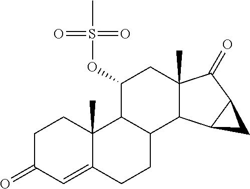

23 ml of mesyl chloride were added dropwise to a solution of 22 g of 11α-hydroxy-15β,16β-methyleneandrost-4-ene-3,17-dione in 220 ml of pyridine at 0° C., and the mixture was stirred at 25° C. for 2 hours. It was then diluted with ethyl acetate, washed with sodium bicarbonate solution, water and brine until neutral, dried over sodium sulphate and concentrated in vacuo at 40° C. 24.7 g of 11α-mesyloxy-15β,16β-methyleneandrost-4-ene-3,17-dione were obtained.

¹H-NMR (600 MHz, CDCl3): δ=5.81(m, 1H), 5.09(m, 1H), 1.39 (s, 3H), 1.21 (m,1H) 1.06 (s, 3H).

### c) 15β,16β-Methyleneandrosta-4,9(21)-diene-3,17-dione

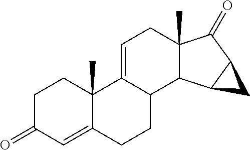

0.82 ml of acetic anhydride was added to a solution of 25.6 g of 11α-mesyloxy-15β,16β-methyleneandrost-4-ene-3,17-dione in 80 ml of acetic acid at 25° C., and the mixture was stirred at a bath temperature of 100° C. for 8 hours. This was followed by addition to water, extraction three times with ethyl acetate, washing with water and brine until neutral, drying over sodium sulphate, and concentrating in vacuo at 40° C. Crystallization from ethyl acetate resulted in 16.4 g of 15β,16β-methyleneandrosta-4,9(11)-diene-3,17-dione.

¹H-NMR (600 MHz, CDCl3): δ=5.79(m, 1H), 5.55(m, 1H), 1.85(m,1H), 1.65(m,1H), 1.37(s, 3H), 1.12-1.33 (2m, 2H), 1.00 (s, 3H).

### d) 9α-Bromo-11β-fluoro-15β,16β-methyleneandrost-4-ene-3,17-dione

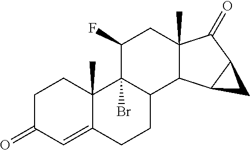

24.5 ml of 70% strength HF/pyridine were slowly added to a suspension of 8.76 g of dibromohydantoin in 250 ml of dichloromethane. 16.3 g of 15β,16β-methyleneandrosta-4,9(11)-diene-3,17-dione were introduced into the resulting solution and stirred at room temperature for 30 min. This was followed by pouring into a mixture of 200 ml of aqueous ammonia (25%) and 300 ml of ice, extracting three times with ethyl acetate, washing with water and brine until neutral, drying over sodium sulphate and concentrating in vacuo at 40° C. Crystallization of the residue from ethyl acetate resulted in 15.2 g of 9α-bromo-11β-fluoro-15β,16β-methyleneandrost-4-ene-3,17-dione.

¹H-NMR (600 MHz, CDCl3): β=5.81(m, 1H) 5.28(dt, 1H), 1.695(d, 3H), 1.175 (d, 3H).

### e) 11β-Fluoro-15β,16β-methyleneandrost-4-ene-3,17-dione

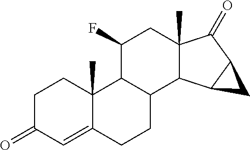

A solution of 33.5 g of 9α-bromo-11β-fluoro-15β,16β-methyleneandrost-4-ene-3,17-dione in 480 ml of benzene was stirred with 42 ml of tributyltin hydride and 416 mg of azobisisobutyronitrile at 80° C. for 30 min. The mixture was concentrated in vacua, and the residue was chromatographed on silica gel 60. Crystallization from ethyl acetate resulted in 18.7 g of 11β-fluoro-15β,16β-methyleneandrost-4-ene-3,17-dione.

¹H-NMR (600 MHz, CDCl3): β=5.73(m, 1H), 5.28(dq, 1H), 1.395(d, 3H), 1.175 (d, 3H).

### f) 11β-Fluoro-3-methoxy-15β,16β-methyleneandrosta-3,5-diene-17-one

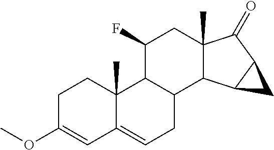

1.3 g of pyridine tosylate were introduced into a suspension of 10.79 g of 11β-fluoro-15β,16β-methyleneandrost-4-ene-3,17-dione in 220 ml of 2,2-dimethoxypropane. It was then stirred at a bath temperature of 100° C. for 3 h. After cooling to room temperature, 2.5 ml of triethylamine were added, and the mixture was concentrated to dryness in vacuo. The residue was stirred with 30 ml of methanol and filtered off with suction. 9.6 g of 11β-fluoro-3-methoxy-15β,16β-methyleneandrosta-3,5-diene-17-one were obtained.

¹H-NMR (600 MHz, CDCl3): δ=5.2.7-5.19 (m, 1.5H), 5,14 (m, 1H), 5.08(q, 0.5 H), 3.60(s, 3H), 1.17 (m, 6H).

### g) 11β-Fluoro-3-methoxy-15β,16β-methylene-17-pregna-3,5-diene-21,17β-carbolactone

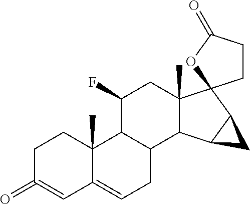

14 g of allyl tetramethylphosphorodiamidate dissolved in 30 ml of tetrahydrofuran were added dropwise to 91 ml of 1.6 M butyllithium solution (in hexane) at −50° C. After stirring at −20° C. for 30 min, 22 ml of N,N,N,N-tetramethylethanediamine were introduced, and the mixture was allowed to warm to room temperature. A solution of 15 g of 11β-fluoro-3-methoxy-15β,16β-methyleneandrosta-3,5-diene-17-one in 80 ml of tetrahydrofuran was added, and the mixture was stirred at room temperature for 4 hours. This was followed by adding saturated aqueous ammonium chloride solution, and pouring into water, extracting three times with ethyl acetate, washing with water and brine until neutral, drying over sodium sulphate, and concentrating in vacuo at 40° C. Crystallization from ethyl acetate resulted in 15.8 g of 11β-fluoro-3-methoxy-15β,16β-methylene-17-pregna-3,5-diene-21,17β-carbolactone.

¹H-NMR (300 MHz, CDCl3): δ=5.28-5.22 (m, 1.5H) 5.17 (m, 1H), 5.09 (q, 0.5 H), 3.63 (s, 3H), 1.20 (m, 6H), 0.53 (m, 1H)

### h) 11β-Fluoro-15β,16β-methylene-3-oxo-17-pregna-4,6-diene-21,17β-carbolactone

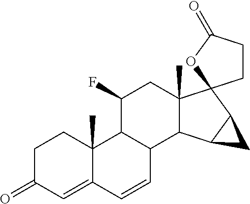

14.5 ml of a 10% strength sodium acetate solution and 5.11 g of 1,3-dibromo-5,5-dimethylhydantoin were successively added in portions at 0° C. to a suspension of 13.5 g of 11β-fluoro-3-methoxy-15β,16β-methylene-17-pregna-3,5-diene-21,17β-carbolactone in 150 ml of 1-methyl-2-pyrrolidone. The mixture was then stirred at 0° C. (ice bath) for 0.5 hours and, after addition of 4.86 g of lithium bromide and 4.27 g of lithium carbonate, stirred at a bath temperature of 100° C. for 3.5 hours. It was then poured into ice-water/sodium chloride, and the precipitate was filtered off. Chromatography on silica gel 60 (elution with hexane/ethyl acetate 1:1) resulted in 9.1 g of 11β-fluoro-15β,16β-methylene-3-oxo-17-pregna-4,6-diene-21,17β-carbolactone.

¹H-NMR (600 MHz, CDCl3): δ=6.41 (m, 1H), 6.22 (m, 1H), 5.68 (s, 1H), 5.11 (dq, 1H), 1.31 (d, 3H), 1.21(d, 3H), 0.60 (m, 1H).

### i) 6β,7β;15β,16β-Dimethylene-11β-fluoro-3-oxo-1 7-progn-4-ene-21,17β-carbolactone

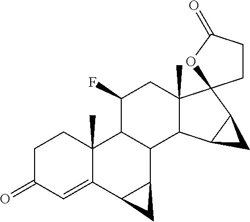

2.39 g of sodium hydride (60% in mineral oil) were added in portions to a solution of 13.41 g of trimethylsulphoxonium iodide in 250 ml of dry DMSO at room temperature and, after addition was complete, the mixture was stirred at room temperature for 3 hours. Then 8.38 g of 11β-fluoro-15β,16β-methylene-3-oxo-17-pregna-4,6-diene-21,17β-carbolactone were introduced, and the mixture was stirred at room temperature for 6 hours. This was followed by pouring into water, extracting three times with ethyl acetate, washing with water and brine until neutral, drying over sodium sulphate and concentrating in vacuo at 40° C. Purification was by chromatography on silica gel 60 (elution with hexane ethyl acetate 1:4). 2.6 g of 6β,7≢2;15β,16β-dimethylene-11β-fluoro-3-oxo-17-pregn-4-ene-21,17β-carbolactone were obtained as fraction A.

MS (EI): m/z=384 (M⁺), 349, 273, 260;

¹H-NMR (600 MHz, CDCl3): δ=5.99 (s, 1H), 5.07 (d(br), 1H), 2.69-2.61 (m, 2H), 2.53 (m, 1H), 2.43 (d(br), 1H), 2.35 (m, 1H), 2.27 (m, 1H), 2.17-2.10 (m, 2H), 2.02-1.95 (m, 2H), 1.83 (m, 1H), 1.68-1.62 (m, 2H), 1.61-1.52 (m, 2H), 1.49 (m, 1H), 1.40 (m, 1H), 1.29 (d, 3H), 1.25 (m, 1H), 1.21 (m, 1H), 1.15 (d, 3H), 1.04 (m, 1H), 0.59 (m, 1H)

### Alternative method for synthesizing Example 2

### c′) 15β,16β-Methyleneandrosta-4,9(11)-diene-3,17-dione and e′) 11β-fluoro-15β,16β-methyleneandrost-4-ene-3,17-dione

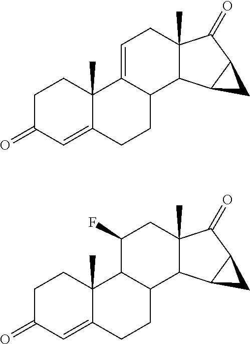

0.47 ml of 1,8-diazabicyclo[5.4.0]undec-7-ene (1.5-5) was added dropwise to a solution of 630 mg of 11α-hydroxy-15β,16β-methyleneandrost-4-ene-3,17-dione in 16 ml of tetrahydrofuran at 0° C. in such a way that the internal temperature did not exceed 5° C. The mixture was then stirred at 0° C. for 30 min., 0:55 ml of perfluorobutane-1-sulphonyl fluoride was added dropwise in such a way that the internal temperature did not exceed 5° C., and the mixture was stirred at 0° C. for a further 1.5 hours. It was then diluted with ethyl acetate, washed with 2M sulphuric acid, saturated sodium bicarbonate solution and water, dried over sodium sulphate and concentrated in vacuo at 40° C. Chromatography on silica gel resulted after elution with hexane/ethyl acetate (1:1) in 156,166-methylene-androsta-4,9(11)-diene-3,17-dione as fraction 1.

1H-NMR (600 MHz, CDCl3): δ=5.79(m, 1H), 5.55(m, 1H), 1.85(m,1H), 1.65(m,1H), 1.37(s, 3H), 1.12-1.33 (2m, 2H), 1.00 (s, 3H)

11β-Fluoro-15β,16β-methyleneandrost-4-ene-3,17-dione is isolated as fraction 2.

¹H-NMR (600 MHz, CDCl3): δ=5.73(m, 1H), 5.28(dq, 1H), 1.395(d, 3H), 1.175 (d, 3H).

### EXAMPLE 3

### 6α,7α;15β,16β-Dimethylene-11β-fluoro-3-oxo-17-pre n-4-ene-21,17β-carbolactone

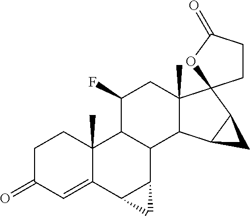

0.37 g of 6α,7α;15β,16β-dimethylene-11β-fluoro-3-oxo-17-pregn-4-ene-21,17β-carbolactone was obtained as fraction B of Example 2.

MS (EI): m/z=384 (M⁺);

¹H-NMR (600 MHz, CDCl3): δ=5.94 (s, 1H), 5.08 (d(br), 1H), 1.35 (s, 3H), 1.25 (m, 1H), 1.21 (m, 1H), 1.20 (d, 3H), 1.00 (m, 1H), 0.76 (ddd, 1H), 0.54 (m, 1H) 0.48 (m, 1H)

### Alternative Methods for Synthesizing Example 1

### 1st Variant

### a. 11α-Hydroxy-3-methoxy-15β,16β-methyleneandrost-3,5-dien-17-one

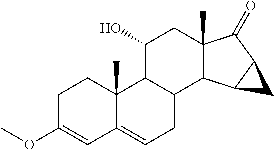

3.2 g of pyridine tosylate were introduced into a suspension of 27 g of 11α-hydroxy-15β,16β-methyleneandrost-4-ene-3,17-dione in 422 ml of 2,2-dimethoxypropane. The mixture was then stirred at a bath temperature of 100° C. for 18 h. Cooling to room temperature was followed by addition of 10 ml of triethylamine and concentration to dryness in vacuo. The residue was stirred with 60 ml of methanol and filtered off with suction. 14.3 g of 11α-hydroxy-3-methoxy-15β,16β-methyleneandrost-3,5-dien-17-one were obtained.

¹H-NMR (400 MHz, CDCl3): δ=5.33 (d, broad, J=3.8Hz, 1H), 5.14 (s, broad, 1H), 4.07 (m, 1H), 3.58 (s, 3H), 1.79 (m, 1H), 1.13 (s, 3H), 1.02 (s, 3H)

### b) 11α-Hydroxy-3-methoxy-15β,16β-methylene-17-pregna-3,5-diene-21,17β-carbolactone

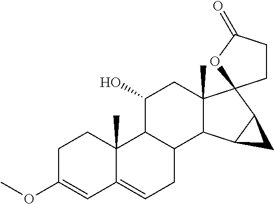

10.24 g of allyl tetramethylphosphorodiamidate, dissolved in 13 ml of tetrahydrofuran, were added dropwise to 66.6 ml of 1.6M butyllithium solution (in hexane) at −50° C. After stirring at −20° C. for 30 min. 16 ml of N,N,N′,N′-tetramethylethanediamine were introduced, and then a solution of 5 g of 11α-hydroxy-3-methoxy-15β,16β-methylene-androst-3,5-dien-17-one in 33.5 ml of tetrahydrofuran was added dropwise. The mixture was warmed to room temperature and then stirred for 30 minutes. This was followed by adding 25 ml of saturated aqueous ammonium chloride solution, and pouring into water, extracting three times with ethyl acetate, washing with water and brine until neutral, drying over sodium sulphate, and concentrating in vacuo. Crystallization from diisopropyl ether resulted in 2.85 g of 11α-hydroxy-3-methoxy-15β,16β-methylene-17-pregna-3,5-diene-21,17β-carbolactone.

¹H-NMR (400 MHz, CDCl3): δ=5.31 (d, broad, J=4.0 Hz, 1H), 5.14 (s, broad, 1H), 4.06 (m, 1H), 3.58 (s, 3H), 1.14 (s, 3H), 1.02 (s, 3H), 0.46 (m, 1H)

### c) 11α-Hydroxy-15β,16β-methylene-3-oxo-17-pegna-4,6-diene-21,17β-carbolactone

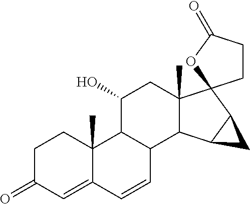

14.8 ml of a 10% strength sodium acetate solution and 4.g of 1,3-dibromo-5,5-dimethyl-hydantoin were successively added in portions at 0° C. to a suspension of 13.5 g of 11α-hydroxy-3-methoxy-15β,16β-methylene-17-pregna-3,5,9(11)-triene-21,17β-carbolactone in 144 ml of 1-methyl-2-pyrrolidone. The mixture was then stirred at 0° C. (ice bath) for 0.5 hours and, after addition of 4.88 g of lithium bromide and 4.31 g of lithium carbonate, stirred at a bath temperature of 80° C. for 3 hours. It was then poured into ice-cold saturated aqueous sodium chloride solution and extracted with ethyl acetate, and the organic phase was washed with water and saturated aqueous sodium chloride solution, dried over sodium sulphate and filtered, and the filtrate was concentrated to dryness. 12.8 g of 11α-hydroxy-15β,16β-methylene-3-oxo-17-pregna-4,6-diene-21,17β-carbolactone were obtained as crude product. A sample was chromatographed on silica gel with a mixture of hexane and ethyl acetate for analytical purposes.

¹H-NMR (400 MHz, CDCl3): δ=6.34 (d, broad, J=9.6 Hz, 1H), 6.20 (d, broad, J=9.6 Hz, 1H), 5.71 (s, broad, 1H), 4.05 (m, 1H), 1.95 (m, 1H), 1.85 (m, 1H), 1.29 (m, 1H), 1.25 (s, 3H), 1.09 (s, 3H), 0.57 (m, 1H)

### d) 11α-Mesyloxy-15β,16β-methylene-3-oxo-17-pregna-4,6-diene-21,17β-carbolactone

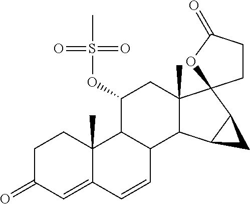

12.8 g of 11α-hydroxy-15β,16β-methylene-3-oxo-17-pregna-4,6-diene-21,17β-carbolactone were dissolved in 113 ml of pyridine. 10.91 ml of methanesulphonyl chloride were then added dropwise. The mixture was stirred at room temperature for 90 minutes and poured into 1.51 of ice-water. Stirring for two hours was followed by filtration with suction, and the filter cake was dried and chromatographed on silica gel with a mixture of hexane and ethyl acetate. 5.4 g of 11α-mesyloxy-15β,16β-methylene-3-oxo-17-pregna-4,6-diene-21,17β-carbolactone were obtained.

¹H-NMR (400 MHz, CDCl3): δ=6.33 (d, broad, J=9.6 Hz, 1H), 6.23 (d, broad, J=9.6 Hz, 1H), 5.74 (s, broad, 1H), 5.10 (m, 1H), 3.01 (s, 3H), 1.56 (m, 1H), 1.45 (m, 1H), 1.30 (s, 3H), 1.14 (s, 3H), 0.59 (m, 1H)

### e) 15β,16β-Methylene-3-oxo-17-pregna-4,6,9(11)-triene-21,17β-carbolactone

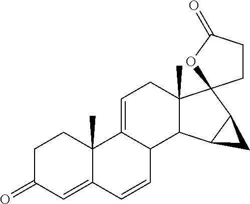

14.8 ml of acetic acid, 0.16 ml of acetic anhydride and 2.44 g of sodium acetate were stirred at 90° C. until the sodium acetate had dissolved. 5.3 g of 11α-mesyloxy-15β,16β-methylene-3-oxo-17-pregna-4,6-diene-21,17β-carbolactone were added to this solution. Stirring at 100° C. for 5 hours was followed by pouring into ice-water and extraction three times with ethyl acetate. Washing of the organic phase with water and saturated aqueous sodium chloride solution, and drying over magnesium sulphate were followed by filtration and concentration of the filtrate. Chromatography on silica gel with a mixture of hexane and ethyl acetate resulted in 2.12 g of 15β,16β-methylene-3-oxo-17-pregna-4,6,9(11)-triene-21,17β-carbolactone.

¹H-NMR (300 MHz, CDCl3): δ=6.36 (d, broad, J=9.6 Hz, 1H), 6.24 (d, broad, J=9.6 Hz, 1H), 5.72 (s, broad, 1H), 5.48 (m, 1H), 3.09 (d, broad, J=11.7 Hz, 1H), 1.84 (m, 1H), 1,47 (m, 1H), 1.38 (m, 1H), 1.32 (s, 3H), 1.03 (s, 3H), 0.59 (m, 1H)

### f) 6β,7β;15β,16β-Dimethylene-3-oxo-17-pregna-4,9(11)-diene-21,17β-carbolactone

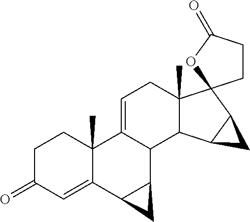

0.09 g of sodium hydride (60% in mineral oil) was added in portions to a solution of 0.52 g of trimethylsulphoxonium iodide in 4 ml of dry DMSO at room temperature and, after addition was complete, the mixture was stirred at room temperature for 2 hours. Then, at 0° C., 0.2 g of 15β,16β-methylene-3-oxo-17-pregna-4,6,9(11)-triene-21,17β-carbolactone was introduced, and the mixture was stirred at room temperature for 2.5 hours. The mixture was then stirred into 100 ml of sulphuric acid (8 per cent by volume) and extracted with ethyl acetate. The organic phase was washed successively with water and saturated sodium chloride solution, dried over sodium sulphate and filtered. Concentration in vacuo and chromatography on silica gel with an eluent composed of ethyl acetate and hexane resulted in 30 mg of 6β,7β;15β,16β-dimethylene-3-oxo-17-pregna-4,9(11)-diene-21,17β-carbolactone.

For spectroscopic data, cf. Example 1b.

2nd Variant

### a. 3-Methoxy-15β,16β-methyleneandrost-3,5,9(11)-triene-17-one

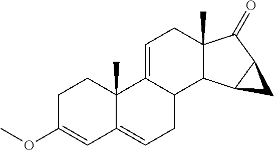

0.8 g of pyridine tosylate was introduced into a suspension of 6.4 g of 15β,16β-methyleneandrost-4,6,9(11)-triene-3,17-dione in 106 ml of 2,2-dimethoxypropane. The mixture was then stirred at a bath temperature of 100° C. for 6 h. Cooling to room temperature was followed by addition of 5 ml of pyridine and, after 5 minutes, concentration to dryness in vacua. The residue was stirred with 130 ml of methanol and filtered off with suction. 4.15 g of 3-methoxy-15β,16β-methyleneandrost-3,5,9(11)-triene-17-one were obtained.

¹H-NMR (400 MHz, CDCl3): δ=5.47 (s, broad, 1H), 5.33 (s, broad, 1H), 5.19 (s, broad, 1H), 3.59 (s, 3H), 2.70 (m, 2H), 2.38 (m, 1H), 1.83 (m, 1H), 1.66 (m, 1H), 1.15 (s, 3H), 0.99 (s, 3H)

### b) 3-Methoxy-15β,16β-methylene-17-pregna-3,5,9(11)-triene-21,17β-carbolactone

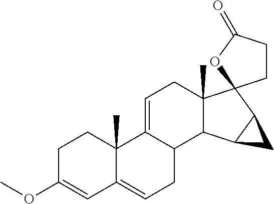

6.51 g of allyl tetramethylphosphorodiamidate, dissolved in 11.4 ml of tetrahydrofuran, were added dropwise to 42.2 ml of 1.6M butyllithium solution (in hexane) at −50° C. After stirring at −20° C. for 30 min, 10.21 ml of N,N,N′,N′-tetramethylethanediamine were introduced, and then a solution of 4.14 g of 3-methoxy-15β,16β-methyleneandrost-3,5,9(11)-triene-17-one in 29.4 ml of tetrahydrofuran was added dropwise. The mixture was warmed to room temperature and stirred for 30 minutes. This was followed by addition of 21 ml of saturated aqueous ammonium chloride solution and pouring into water, extracting three times with ethyl acetate, washing with water and brine until neutral, drying over sodium sulphate, and concentrating in vacuo, whereupon crystallization started. Filtration with suction to remove remaining solvent resulted in 3.14 g of 3-methoxy-15β,16β-methylene-3,5,9(11)-triene-21,17β-carbolactone.

¹H-NMR (300 MHz, CDCl3): δ=5.45 (s, broad, 1H), 5.32 (s, broad, 1H), 5.18 (s, broad, 1H), 3.59 (s, 3H), 1.15 (s, 3H), 0.96 (s, 3H)

### c) 15β,16β-Methylene-3-oxo-17-pregna-4,6,9(11)-triene-21,17β-carbolactone

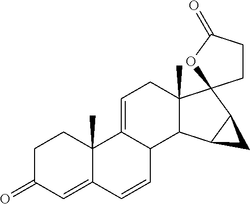

1.7 ml of a 10% strength sodium acetate solution and 0.6 g of 1,3-dibromo-5,5-dimethylhydantoin were successively added in portions at 0° C. to a suspension of 2.2 g of 3-methoxy-15β,16β-methylene-17-pregna-3,5,9(11)-triene-21,17β-carbolactone in 35 ml of 1-methyl-2-pyrrolidone. The mixture was then stirred at 0° C. (ice bath) for 0.5 hours and after addition of 0.83 g of lithium bromide and 0.74 g of lithium carbonate, stirred at a bath temperature of 100° C. for 3.5 hours. It was then poured into ice-water/sodium chloride, and the precipitate was filtered off. Chromatography on silica gel 60 (elution with hexane/ethyl acetate 1:1) resulted in 1.2 g of 15β,16β-methylene-3-oxo-17-pregna-4,6,9(11)-triene-21,17β-carbolactone.

For spectroscopic data, compare 1st variant e

### d) 6β,7β;15β,16β-Dimethylene-3-oxo-17-pregna-4,9(11)-diene-21,17β-carbolactone

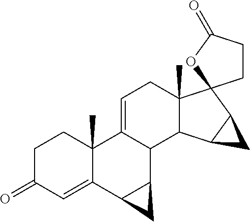

For procedure and working up, cf. 1st variant f.

1. Pregn-4-ene-21,17-carbolactones of the general formula I 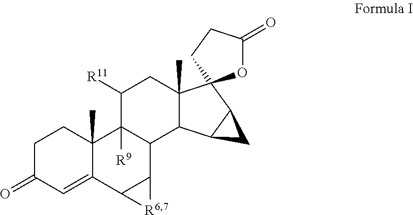
in which
R^{6,7} is an α- or β-methylene and
R⁹ is a hydrogen atom and R¹¹ is a bromine, chlorine or fluorine atom or
R⁹ and R¹¹ together are a bond.

2. Pregn-4-ene-21,17-carbolactones according to claim 1, characterized in that R⁹ is located in the α position.

3. Pregn-4-ene-21,17-carbolactones according to claim 1, characterized in that R¹¹ is located in the β position.

4. Pregn-4-ene-21,17-carbolactones according to claim 1, characterized in that the halogen atom R¹¹ is a fluorine or chlorine atom.

5. Pregn-4-ene-21,17-carbolactones according to claim 4, characterized in that the halogen atom R¹¹ is a fluorine atom.

6. 6β,7β;15β,16β-Dimethylene-3-oxo-17-pregna-4,9(11)-diene-21,17β-carbolactone according to claim 1.

7. Pregn-4-ene-21,17-carbolactones according to claim 1, specifically 11β-chloro-6β,7β;15β,16β-dimethylene-3-oxo-17-pregn-4-ene-21,17β-carbolactone 6β,7β;15β,16β-dimethylene-11β-fluoro-3-oxo-17-pregn-4-ene-21,17β-carbolactone 6α,7α;15β,16β-dimethylene-11β-fluoro-3-oxo-17-pregn-4-ene-21,17β-carbolactone.

8. Pharmaceutical products comprising at least one compound of the general formula I according to claim 1, and a pharmaceutically acceptable carrier.

9. Pharmaceutical products according to claim 8, comprising 6β,7β;15β,16β-dimethylene-3-oxo-17-pregna-4,9(11)-diene-21,17β-carbolactone.

10. Pharmaceutical products according to claim 8, comprising 6β,7β;15β,16β-dimethylene-11β-fluoro-3-oxo-17-pregn-4-ene-21,17β-carbolactone.

11. Pharmaceutical products according to claim 8, additionally comprising at least one oestrogen.

12. Pharmaceutical products according to claim 11, comprising ethinylestradiol.

13. Pharmaceutical products according to claim 11, comprising a natural oestrogen.

14. Pharmaceutical products according to claim 13, comprising oestradiol.

15. Pharmaceutical products according to claim 13, comprising oestradiol valerate.

16. Pharmaceutical products according to claim 13, comprising at least one conjugated oestrogen.

17. 11α-Hydroxy-15β,16β-methyleneandrost-4-ene-3,17-dione as a starting compound for preparing the compounds of the general formula I.

18. Process for preparing 11α-hydroxy-15β,16β-methyleneandrost-4-ene-3,17-dione, characterized in that 15β,16β-methyleneandrost-4-ene-3,17-dione is hydroxylated in a fermentor with microorganisms of the species Absidia sp., Acremonium sp., Ascochyta sp., Aspergillus sp., Bacillus sp., Beauveria sp., Botryodipoldia sp., Caldariomyces sp., Calonectria sp., Colletotrichum sp., Curvularia sp., Fusarium sp., Gibberella sp., Gloeosporium sp., Glomerella sp., Gnomonia sp., Haplosporella sp., Helicostylum sp., Helminthosporium sp., Metarhizium sp., Mucor sp., Nigrospora sp., Rhizopus sp., Sporotrichum sp., Syncephalastrum sp., and Wojnowicia sp.

19. Process according to claim 18, characterized in that hydroxylation is carried out with Absidia orchidis, Absidia coerulea, Acremonium strictum, Ascochyta clematidina, Aspergillus alliaceus, Aspergillus awamori, Aspergillus fischeri, Aspergillus flavus, Aspergillus malignus, Aspergillus melleus, Aspergillus nidulans, Aspergillus niger, Aspergillus ochraceus, Aspergillus variecolor, Bacillus megaterium, Beauveria bassiana, Beauveria tenella, Botryodiplodia malorum, Caldariomyces fumago, Calonectria decora, Colletotrichum phomoides, Curvularia lunata, Fusarium oxysporium, Fusarium solani, Gibberella zeae, Glomerella cingulata, Gloeosporium fructigenurn, Gloeosporium higgensianum, Gloeosporium kaki, Gloeosporium lacticolor, Gloeosporium olivarum, Glomerella fusaroides, Gnomonia cingulata, Haplosporella hesperedica, Helminthosporium sp., Helicostylum piriforme, Metarhizium anisopliae, Mucor plumbeus, Mucor spinosus, Nigrospora sphaerica, Rhizopus arrhizus, Rhizopus cohnii, Rhizopus delemar, Rhizopus japonicus, Rhizopus kazaensis, Rhizopus microsporus, Rhizopus oryzae, Rhizopus shanghaiensis, Rhizopus stolonifer, Rhizopus tritici, Sporotrichum sulfurescens, Syncephalastrum racemosum, Wojnowicia graminis and Wojnowicia hirta.

20. Process according to claim 19, characterized in that hydroxylation is carried out with Absidia orchidis (ATCC 6647), Acremonium strictum (NRRL 5759), Ascochyta clematidina (CBS), Aspergillus alliaceus (ATCC 10060), Aspergillus awamori (CBS), Aspergillus fischeri (ATCC 1020), Aspergillus malignus (IMI 16061), Aspergillus melleus (CBS), Aspergillus nidulans (ATCC 11267), Aspergillus niger (ATCC 9142, ATCC 11394), Aspergillus ochraceus (NRRL405, NRRL 410, CBS 13252, ATCC 46504), Aspergillus variecolor (ATCC 10067), Bacillus megaterium (ATCC 13368), Beauveria bassiana (IFO 5838, ATCC 13144, IFO 4848, CBS 11025, CBS 12736, ATCC 7159), Botryodiplodia malorum (CBS 13450), Caldariomyces fumago (ATCC 16373), Calonectria decora (ATCC 14767), Curvularia lunata (IX 3, NRRL 2380), Fusarium solani (ATCC 12823), Fusarium oxysporum (ATCC 7808), Gibberella zeae (CBS 4474), Glomerella cingulata (ATCC 12097, ATCC 10534, CBS 23849, CBS 23749, ATCC 16646, IFO 6459, IFO 6425, IFO 6470, ATCC 15093, ATCC 10529, IFO 5257, ATCC 56596, ATCC 64682), Glomerella fusaroides (ATCC 9552), Gnomonia cingulata (CBS 15226), Haplosporella hesperedica (CBS 20837), Helicostylum piriforme (ATCC 8992), Helminthosporium sp. (NRRL 4671), Metarhizium anisopliae (IFO 5940), Mucor plumbeus (CBS 29563), Nigrospora sphaerica (ATCC 12772), Rhizopus arrhizus (ATCC 11145), Rhizopus oryzae (ATCC 4858, ATCC 34102, CBS 32947), Rhizopus stolonifer (ATCC 15441), Syncephalastrum racemosum (IFO 4827) and Wojnowicia graminis (CBS 89168).
